(19) United States
(12) Patent Application Publication
(10) Pub. No.: US 2021/0268136 A1
(43) Pub. Date: Sep. 2, 2021

(54) PROCESS AND SYTEM FOR ULTRASONIC DRY MIST DISPENSER AND OZONE SANITIZER

(71) Applicants: Eric Engler, Pompano Beach, FL (US); David Silverman, Indian Harbor Beach, FL (US)

(72) Inventors: Eric Engler, Pompano Beach, FL (US); David Silverman, Indian Harbor Beach, FL (US)

(21) Appl. No.: 17/320,691

(22) Filed: May 14, 2021

Related U.S. Application Data
(60) Provisional application No. 62/025,649, filed on Jul. 17, 2014.

(51) Int. Cl. A61L 2/22; B05B 17/06; B05B 9/01; B05B 9/00; A61L 9/14

(52) U.S. Cl. CPC A61L 2/22; B05B 17/0615; B05B 9/01; B05B 9/007; A61L 2209/132; A61L 2202/11; A61L 2202/14; A61L 2202/16; A61L 9/14

(57) ABSTRACT

A process for sanitizing and/or disinfecting porous and solid surfaces and airborne particles in ambient air by killing bacteria and viruses, including the steps of: providing an enclosed water storage tank having a predetermined maximum water surface level, generating ultrasonically with an ultrasonic oscillator mounted slightly below the water level in the water storage tank, a dry mist that is forced into a blending chamber above said water level in said water storage tank; an air blower with an airflow outlet connected into said water storage tank, and above the tank maximum water level, and an ozone generator outlet connected into said water storage tank for providing a predetermined amount of ozone, above the water level, into the water storage tank during operation, creating an ozone-saturated dry mist. The ozone dissolves in the dry mist and forms a saturated or super-saturated dry mist created in the water storage tank. The system can be mounted on a mobile platform for manually positioning the mixture delivery system.

### CROSS REFERENCE TO RELATED APPLICATIONS

This application claims the benefit of provisional U.S. Patent Application Ser. No. 63/025,649, filed on May 15, 2021.

### BACKGROUND OF THE INVENTION

### 1. Field of the Invention

This invention relates generally to a process for generating a dry mist saturated with dissolved ozone and dispensing the dry mist saturated with dissolved ozone for sanitizing hard and soft surfaces and airborne particles, and a system having an ultrasonic oscillator to dispense the ozone-saturated dry mist to sanitize and/or disinfect hard and soft surface areas and airborne particles. Airborne particles may have bacteria and/or viruses on them or may actually be bacteria and/or viruses.

The recent worldwide pandemic of the COVID-19 virus has created an urgent demand for a sanitization and/or disinfection process and system that can disinfect and sanitize hard and soft surfaces and airborne particles in a fast and efficient manner to remove bacteria and viruses.

Numerous processes and systems using liquid disinfectants that are typically sprayed onto surface areas and into ambient air, are well known in the art, but can have deleterious effects on people, because liquid disinfectants can leave toxic chemicals on surfaces or create toxic vapors in the surrounding air. Thus, liquid disinfectants may affect exposed persons, causing various health disorders.

This invention uses pure oxygen to prevent issues of other ozone-generating machines that rely on introducing ozone gas into the ambient air, which is inadequate for the quick and safe dispersion of ozone molecules, due to the harmful side effects of ozone gas. Because ambient-air, ozone-gas-generating machines utilize ambient air to produce ozone, and the air can contain copious amounts of nitrogen and humidity, it results in air surfaces that can include nitric oxides and nitric acid.

The present invention process and system provides a highly effective, fine, dry mist that is slightly super-saturated with ozone sanitizer and/or disinfectant that can be quickly and efficiently distributed and dispensed onto surfaces and airborne particles, producing a high sanitizing and/or disinfection rate of viruses and bacteria, with no harmful residual surface chemicals to negatively affect a person's health.

### SUMMARY OF THE INVENTION

A process that includes the steps to ultrasonically generate a dry water mist saturated with ozone, and dispense the dry mist saturated with ozone onto hard and soft surfaces and airborne particles for disinfecting hard and soft surfaces to remove bacteria and viruses, without leaving a wet surface. The improved system includes an ultrasonically-generated, substantially dry mist, saturated, super-saturated, or slightly supersaturated with dissolved ozone dispensing and sanitizing and or disinfecting system for hard and soft surfaces and airborne particles. The system comprises an exterior housing, a water storage tank mounted inside said exterior housing, an pure oxygen O₂ supply tank attached to the outside of said exterior housing, and an air blower connected on a side wall of said internal water storage tank. The air blower has an air flow outlet opening into the water storage tank, with the air flow outlet positioned above the surface of a maximum, predetermined level of water stored in the water tank. The air blower outlet into the water storage tank may include an input air flow diverter, directing incoming air downwardly, toward the water surface.

An ozone generator conduit outlet is mounted and opens into the water storage tank, preferably on the water storage tank vertical wall that includes the air blower outlet, and above the water storage tank maximum water surface level, but below the air blower outlet into the water storage tank, so that incoming ozone is directed just above the water surface.

A piezoelectric ultrasonic oscillator that generates a very fine dry mist is positioned inside the water storage tank, preferably on a water float, just below the water surface. Its structure and function are described below.

An elongated, flexible, saturated dry mist ozone dispensing hose, used for ozone sanitizing hard and soft surfaces and airborne particles, is connected by an access fitting hose fastener to an access fitting, namely, a threaded or quick-disconnect opening in the top of the exterior housing. And, also connected internally to the top to an access fitting opening in the top surface of the internal water storage tank. The dispensing hose emits the ozone-saturated dry mist from the upper volume of the water storage tank, above the water surface, dispensing the ozone-saturated dry mist produced in said water storage tank through the dispensing hose onto hard and soft surfaces and airborne particles for disinfecting and sanitizing, without leaving a moist residue on the surfaces.

The top surface of the exterior housing and the top surface of the water storage tank both include second access fittings with openings, each fitting connected to a second conduit to receive inlet water that allows the water storage tank to be filled with water from outside of the water storage tank and from outside of the exterior housing. The inlet water conduit can include a removable, threaded cap at the exterior housing access fitting opening, which is threaded.

The water storage tank may, in some embodiments, be a cube or rectangular prism, with six sides, each rectangular in shape, with four vertical side surfaces, a flat bottom surface, and a substantially top flat surface. The volume of the water in the water storage tank, during operation, is determined by a predetermined maximum water level, the height or depth of the water in the water storage tank, vertically. Two water level sensor lights may be used on the exterior housing door, to show the maximum fill level, when filled with the proper, desired amount of water and a minimum acceptable level. The water storage tank has a specifically predetermined, upper vertical side wall water level position. The water storage tank should not be filled above the certain designated level vertically in the water storage tank. The water storage tank also has a minimal water level for operation, unless one desires to empty the water storage tank, for which there is a drain valve provided at the very bottom of the water tank.

The process of generating a fine water mist and saturating the fine water mist with ozone to create an ozone-saturated dry mist with a predetermined, desired amount of ozone in parts per million, resulting in very efficient sanitizing/disinfection of hard and soft surfaces and airborne particles, without causing surface wetness, is an extremely important aspect of this invention. In addition, the water storage tank water maximum level position is very important in the operation of this invention.

In order to create the proper ratio of ozone O₃ to saturate the dry mist generated by the ultrasonic oscillator, submerged just below the water level surface, such as ½ inch in some embodiments, applicants have determined that the air blower outlet air flow, with a downward air diverter, is positioned above the water storage tank actual water level at all times, so that incoming air can be directed downwardly toward the water surface in the water storage tank, through an air blower outlet opening above the water surface. The air blower outlet opening, extending from the water storage tank side wall exterior mounted air blower, on the inside of the water storage tank, provides an air flow directing surface outlet that forces the air downwardly from the air blower outlet, toward the water surface level. Also mounted in the same water storage tank sidewall, in some embodiments, is the ozone generator outlet, which is positioned closer to the inside water surface level position so that the incoming, flowing ozone will directed just above the water surface level. Applicants have determined that this positioning of the ozone outlet producing the ozone-saturated dry mist in the water storage tank, along with the air blower dispersing air downwardly towards the water, helps to create the proper ratio and mixture of dry mist and ozone to saturate or slightly supersaturate the dry mist with ozone for maximum sanitizing and/or disinfection of surfaces and airborne particles. In some embodiments, 80-90 and up to 150 parts per million of ozone in the ozone-saturated dry mist is desirable for saturation.

In some embodiments, an ultrasonic oscillator in the form of piezoelectric crystal discs produce the dry mist to be ozone-saturated in the water storage tank are strategically positioned in a cylindrical, doughnut-shaped, floating unit with a central, circular support surface area, that is a flat planar support surface containing a plurality of the ultrasonic oscillator discs, horizontally parallel. In some embodiments, 12 ceramic ultrasonic oscillator discs can be used together. The planar surface support board and ultrasonic oscillator discs (supported in the center of the floating doughnut unit) are purposely located just below the surface of the water, at all times, at least one-half an inch in some embodiments, with each ceramic disc at the same level. Each of the ultrasonic oscillator discs is electrically connected to a power source so that all of the ultrasonic oscillator ceramic discs are electrically connected in parallel (voltage) and operated simultaneously. The ultrasonic frequency generated forces water into the dry mist of droplets into the upper portion of the water storage tank blending chamber, above the water level, filling the blending chamber with dry mist, where they are saturated with ozone.

The ozone generator outlet, mounted to the water storage tank vertical wall, has an inlet conduit with operating pressure valves, to the oxygen tank in order to receive pure oxygen O₂ from the oxygen storage tank. The separate oxygen supply tank is physically mounted on the exterior wall or door of the exterior housing. The oxygen supply tank provides pure O₂ oxygen to the ozone generator, an important factor for saturating the dry mist particles with ozone without creating hazardous components. The oxygen tank outlet conduit can include oxygen flow and pressure regulating valves. The ozone generator output flow is injected into the water storage tank, above the water surface, in conjunction with the air blower outlet air flow into the water storage tank. The volume above the water level in the water storage tank, the blending chamber, becomes filled with dry mist to be saturated with ozone. The operation of the air blower aids in dispensing the dry mist becoming saturated with ozone for sanitizing and/or disinfecting into the dispensing hose and onto hard and soft surfaces and airborne particles. The dry mist saturated with ozone is collated into a thoroughly-mixed, extremely-fine-particle, sanitizing and/or disinfecting mixture that becomes super-saturated with ozone, in some embodiments, at 80-90 and up to 150 parts per million of ozone.

The ozone-saturated dry mist, once formed, and then power-dispersed under low pressure from a dispenser hose outlet that dispenses the ozone-saturated dry mist onto surrounding solid and porous surfaces and airborne particles for complete sanitizing and/or disinfecting, does not deposit a wet residue on the surfaces when sanitized, nor leave behind any chemicals or residue

The ultrasonic, ozone-saturated, dry mist ozone sanitizing and/or disinfecting process, in some embodiments, can be provided from a mobile platform, having four wheels and a manual pushing handle, that includes all of the dry mist, oxygen supply, and ozone-mixing components described herein, including the delivery system, so that the portable system can be easily moved from one area enclosure to another area for use.

It is an object of the invention to provide a process and system for generating an ozone-saturated dry mist using an ultrasonic oscillator, saturating the dry mist with a predetermined amount of dissolved ozone that receives the ozone per million parts for the mixture, and dispensing the mixture of ozone-saturated dry mist onto hard and soft surfaces and airborne particles in the ambient air for sanitizing and/or disinfecting surfaces of viruses and bacteria, without wetting the surfaces.

This device places the ozone where it needs to be, not where it does not. The method of saturating and slightly super-saturating the dry mist by balancing the ratios of ozone to dry mist is the crux of the device. Almost all of the ozone stays dissolved in the specific mist particle size so that all of the ozone contacts the medium of sanitizing rather than escaping into the air. This allows hard and soft surfaces along with particles floating in the air to be contacted and sanitized. Therefore, almost all of the ozone is used in the actual sanitizing process and not dissipated to the air. This reduces or eliminates the need for personal protection equipment and OSHA requirements for workers.

### BRIEF DESCRIPTION OF THE DRAWINGS

FIG. 1A shows a schematic, perspective view of the process and system piezoelectric ultrasonic oscillator used in the invention, inside the water storage tank.

FIG. 1B is a perspective view of the exterior housing of a portable embodiment of the invention, showing a portable ultrasonic oscillator, ozone-saturated dry mist dispenser, and ozone sanitizer exterior housing.

FIG. 2 is a perspective view of the portable embodiment shown in FIG. 1B with an end door open, showing an internal water storage tank inside said portable embodiment exterior housing.

FIG. 3 shows an exploded, perspective view of the portable embodiment shown in FIG. 1 and FIG. 2, with the water storage tank exploded upwardly from the exterior housing and also showing the internal water storage tank exploded to illustrate an ultrasonic oscillator ozone-saturated dry mist generator that is utilized inside the water storage tank.

FIG. 4 shows a side elevational view of the portable embodiment of the invention disclosed in FIGS. 1B-3, partially cutaway, exposing a portion of the water storage tank shown in elevation and in cross-section.

### PREFERRED EMBODIMENT OF THE INVENTION

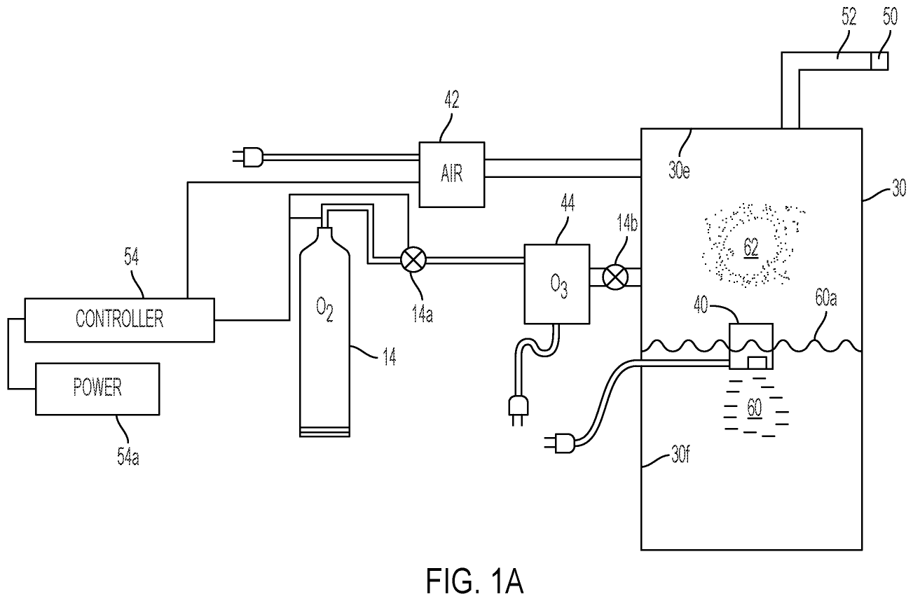

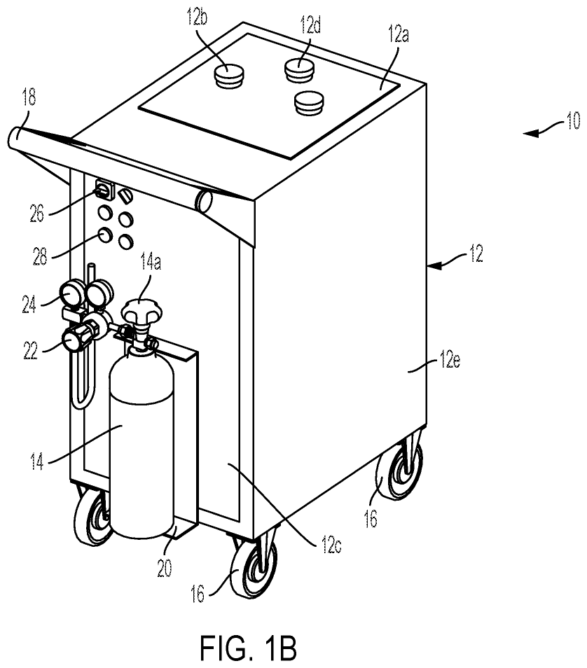

FIG. 1A shows a schematic diagram to of the crux of the invention, which is a process and system to 1) generate an ozone-saturated dry mist in an enclosed water storage tank 30 using an ultrasonic oscillator 40; 2) generate ozone from pure O₂ and mix the ozone with the dry mist to saturate with dissolved ozone, and 3) dispense the ozone-saturated dry mist through a dispensing hose 52 onto hard and soft surfaces and airborne particles to sanitize and/or disinfect bacteria and viruses from the hard and soft surfaces and airborne particles without surface wetting.

To generate dry mist, air from air blower 42 (under low pressure) and ozone from ozone generator 44 are simultaneously directed into the blending chamber 30e in water storage tank 30, above the surface 60a of the water 60. The ultrasonic oscillator 40 generates dry mist that rises into air flow and ozone flow volume ratios to saturate the dry mist blending chamber area 30e completely with and ozone-saturated dry mist. The ozone-saturated dry mist in chamber 30e is directed, under low relative pressure, through a distribution and sanitizer hose 52 and out a nozzle 50 onto a desired hard or soft surface and airborne particles to be sanitized and/or disinfected, without wetting the surface.

An electronic controller 54 is used to operate and manage distribution of the ozone-saturated dry mist sanitizer, and to control the generation of ozone O₃ from the pure O₂ oxygen supply tank 14 supply and pressure valves 14a, and to ensure and maintain saturation quantity and ratios for the sanitizing and/or disinfecting ozone-saturated dry mist for maximum efficiency. Electric power is provided to controller 54 by power unit 54a and for the ultrasonic oscillator 40, the ozone generator 44, and the air blower 42, with inlet plugs.

The air blower air and ozone flow rates contribute to control the amount of ozone to dissolve sufficient ozone to saturate the dry mist in the water storage tank blending chamber 30e to obtain the optimum ratio of dry mist and dissolved ozone for sanitizing and/or disinfecting hard and soft surfaces and airborne particles. In some embodiments, generation and control of air and ozone ratios are the responsibility of sensors and the controller 54 in its operation of all of the essential components of the system.

Referring to FIG. 1B, Applicants' ultrasonic, ozone-saturated dry mist dispenser and ozone sanitizing and/or disinfection system's exterior housing 12 described herein is shown, in one embodiment, as a portable system 10, that includes an exterior housing 12 having three rectangular, vertical sides 12e, a rectangular door 12c that opens outwardly, a bottom panel, a top rectangular panel 12a that includes two threaded access fitting ports 12b and 12d, with caps. One of the access fitting ports 12b is used to allow pure water to be poured to the water storage tank inside the exterior housing 12. The other access fitting 12d is used to connect to a system ozone-saturated dry mist sanitizer dispensing hose (not shown) in FIG. 1B.

The mobile platform exterior housing 12 includes a manual handle 18 and four wheels 16 that allow the system 10 to be manually positioned and moved to different desired locations for sanitizing and disinfecting with a dispensing hose and nozzle, in some embodiments.

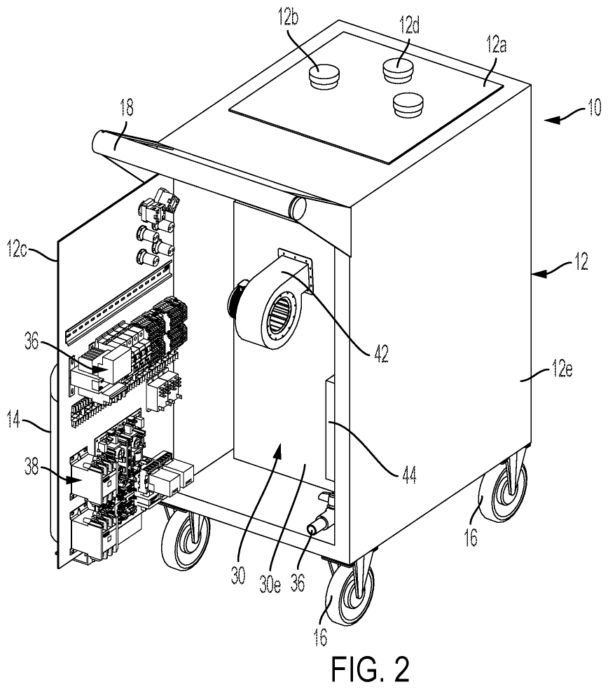

Door 12c supports an oxygen tank 14 that is secured to the door 12c by a support bracket 20 that firmly holds the oxygen tank 14 in place, while being suspended and attached to housing 12. An oxygen tank 14 manual on/off flow valve 14a is provided on oxygen tank 14. Another on/off flow valve 22 and oxygen pressure gauges 24 that can measure the pressure in the oxygen tank 14, while valve 22 allows the oxygen tank to be turned on and off, when providing oxygen to an ozone generator 44, (FIG. 2), as discussed below. Also mounted on door 12c is a system on-off power switch 26, and four status lights 28 that provide status of the system 10 operation, power on and off, the sanitizing operation is “on”, high water level, and minimum water level in the water storage tank. Another status light could show low oxygen O₂.

Referring now to FIG. 2, the system 10 is shown, with door 12c in the open position. The inside of exterior housing 12, and the components and equipment that generate the ozone-saturated dry mist and dispense the ozone-saturated dry mist for sanitizing and/or disinfection are shown.

A major component of the system 10 is a water storage tank 30 that occupies a large volume inside of the exterior housing 12. The water storage tank 30 is substantially a rectangular, six-sided cube or rectangular prism that is used to receive and store pure water. The water, vibrated by an ultrasonic oscillator, becomes the ozone-saturated dry mist used in the invention. The water storage tank 30 has four rigid metal walls that are substantially rectangular, a rectangular floor or base and a rectangular top that has at least two access fittings around ports. One access fitting port is for receiving pure water to be used in the water storage tank and a second access fitting port is for dispensing the ozone-saturated dry mist sanitizer from the water storage tank 30, described in detail below.

In FIG. 2, an air blower 42 and its outlet are attached to one of the vertical water storage tank 30 side walls 30e. An ozone generator 44 is attached physically to the inside of exterior vertical wall 12e, and has an outlet conduit (not shown) attached to the same vertical water storage tank wall 30e as the air blower 42. Thus, air that is blown into the water storage tank 30 from the same vertical side wall 30e as ozone is being directed into the water storage tank 30 from the same side wall 30e is shown below. The water storage tank 30 has sensors (not shown in FIG. 2) to determine the maximum and minimum water levels permitted in the water storage tank so that the incoming air from air blower 32 along with the incoming ozone directed into the water storage tank 30 are above the water surface at all times.

Inside the water storage tank 30 there is an inductive outside float, housing an ultrasonic oscillator (explained below) used to generate the dry mist that gets mixed with incoming air from air blower 42 along with ozone being generated by the ozone generator 44. This operation is discussed in more detail, below. Near the bottom of the water storage tank 30 is a drain and drain valve 34 that allow the water storage tank 30 to be drained by gravity when necessary to remove any undesired water. FIG. 2 shows, mounted on door 12d on the inside, an electrical control unit 36 and an electrical power unit 38, all of which may include the logic circuits and power necessary for controlling the air blower 42, the ozone generator 44 for generating ozone, and operating the dry mist ultrasonic oscillator (that floats inside the water storage tank 30). Electronic circuit operating units 36 and power circuit units 38 also control solenoid valves and the flow rates of the air coming from blower 42 and the dispensing rate of dry mist to achieve the proper proportions of dry mist and ozone to create the ozone-saturated dry mist for use in the system 10, discussed below.

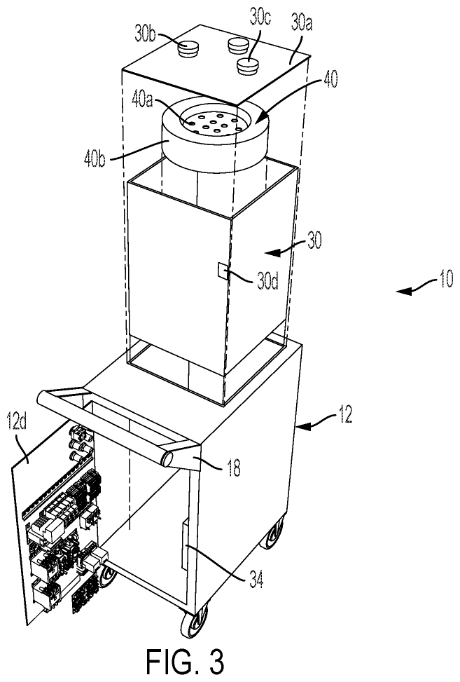

Referring now to FIG. 3, an exploded view of the system 10 is shown. The (exploded} water storage tank 30, is shown elevated, for illustration purposes, above the exterior housing 12. The water storage tank 30 includes an enclosed, six-sided, rectangular prism that includes a rigid metal top surface 30a that has two access fittings and ports 30b and 30c. Access fitting 30b is a port to receive pure water that is deposited into the water storage tank 30 when necessary. Access fitting 30c is a port used to dispense the ozone-saturated dry mist sanitizer and/or disinfectant, generated in the water storage tank 30 mixing chamber 30e. Each access fitting port, 30b and 30c, is connected to the lid (not shown in FIG. 3) to a comparably functioning access fitting port directly above, in exterior housing 12a top panel, shown as access fittings ports 12b and 12d (FIG. 1).

In FIG. 3, the water storage tank 30 contains inside an ultrasonic oscillator 40 (elevated above for illustration purposes). The ultrasonic oscillator 40 is an ultrasonic transducer that generates ultrasonic frequencies from a piezoelectric crystal using ceramic discs submerged in water in the water storage tank to generate dry mist in the water storage tank blending chamber, 30e above the water surface. The water storage tank 30 includes a water-level-sensing device 30d that sets the displays maximum height (or depth) of water in the water storage tank 30. The ultrasonic oscillator 40 includes a float 40b, that is doughnut-shaped in some embodiments. The float 40b is connected to an interior flat panel that includes a plurality of ultrasonic oscillator (frequency-generating) discs 40a that are the transducers that vibrate to generate dry mist in the water while the ceramic discs are submerged below the water, while suspended on float 40b. In some embodiments, applicants have an ultrasonic oscillator with 12 discs 40a mounted on a flat-panel that is connected to a float 40b so that the discs are suspended below the water surface and maintain at least a one-half inch below the water surface at all times to permit the generation of dry mist. The number of ultrasonic piezoelectric oscillator ceramic discs used to generate dry mist, which in one embodiment is twelve, can vary widely, dependent on the water storage tank size 30 and its function. In this embodiment, the invention 10 is a portable unit for sanitizing and/or disinfecting hard and soft surfaces and airborne particles with an ozone-saturated dry mist. The ozone-saturated dry mist ultrasonic oscillator 40 has an electrical connection that provides the proper voltage and amperage to the each individual oscillator disc 40a that vibrates together and are all connected electrically parallel so the ceramic discs 40a all share the same voltage during operation. The ultrasonic oscillator 40, is a dry mist generator to create the dry mist to receive dissolved ozone to create an ozone-saturated dry mist sanitizer and/or disinfectant. The ultrasonic oscillator is also connected to the system electrical operating system that controls its power, on and off, and its operation at all times.

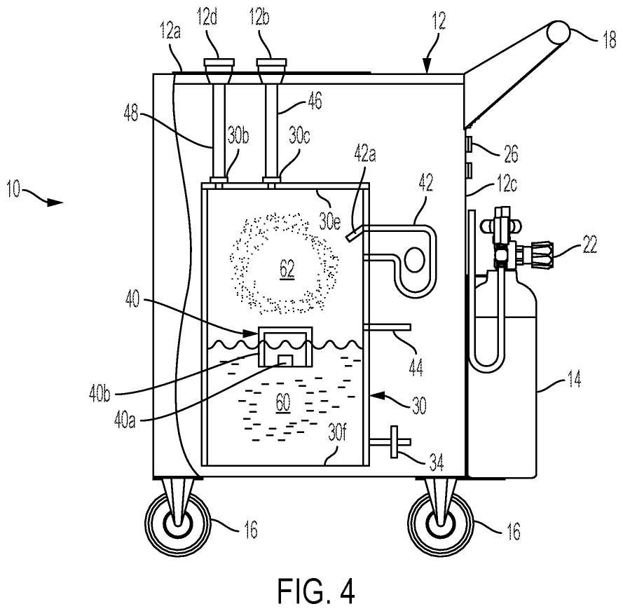

Referring to FIG. 1A and FIG. 4, the method of sanitizing and/or disinfecting hard and soft surfaces and airborne particles in the ambient air to eliminate bacteria and viruses, primarily focuses on creating an ozone-saturated dry mist in the water storage tank 30 using the ultrasonic oscillator 40. An air blower 42 directs air, under low pressure, into the water storage tank 30 and downwardly toward the surface of the water therein, above the level of the ozone entering the water storage tank. The ozone generator 44 generates the ozone that is directed into the water storage tank above the surface of the water, below the incoming air flow from the air blower, to mix with the dry mist being generated by ultrasonic oscillator 40 dry mist generator. The purpose of the invention is to generate a desired ratio of dry mist in union with the ozone, to create an ozone-saturated dry mist, so that when the sanitizer dispenser dispenses the ozone-saturated dry mist under low pressure on a hard or soft surface and airborne particles, the proper ratio is provided for the zone to act to eliminate bacteria and viruses on a hard or soft surface and airborne particles and, at the same time, the ozone-saturated dry mist evaporates from the surface and air quickly, so that a sanitized and/or disinfected surface does not become wet or damp or leave any deleterious residue. Applicants have determined that 80 to 90, and up to 150 parts per million of ozone in the dry mist is very effective as a sanitizer and/or disinfectant for bacteria and viruses on hard and soft surfaces without wetting the surfaces, not leaving a deleterious residue harmful to humans.

Referring now to FIG. 4, the invention system 10 is shown, with a side elevational view of exterior housing 12 cutaway on one side, so that the inside of the water storage tank 30 (viewed in cross-section) is represented schematically in operation, partially filled with water 60. The water storage tank 30 lower volume 30f, contains water 60. Above the water surface in the upper volume blending chamber 30e of water storage tank 30 is an ozone-saturated dry mist 62 that has been generated by the ultrasonic oscillator 40 attached to float 40b on the surface of water 60. Ultrasonic oscillator discs 40a act as transducer frequency generators to generate dry mist 62 in the water storage tank 30, housed in the ultrasonic oscillator 40, and submerged to remain suspended below the water 60 surface. Also shown is the air blower 42 attached to the side wall of water storage tank 30 so that the air directed into the water storage tank upper area (above the water surface) containing the dry mist is directed downwardly by a diverter 42a, forcing the incoming air toward the water surface. Also mounted through the side wall of water storage tank 30 is the ozone generator 44 that is also positioned below the air blower 42 so that incoming ozone from ozone generator 44 is near the surface of the water 60 so that ozone interacts directly with the dry mist coming from the water surface and the downward air directed from air blower 42, creating ozone-saturated dry mist 62 in the upper volume blending chamber 30e of water storage tank 30.

FIG. 4 shows conduits 46 and 48 each attached between the exterior housing panel 12a and the access fitting ports 12b and 12d and the water storage tank 30 access fitting ports 30b and 30c. Conduit 48 is used to supply water internally from the exterior housing 12 through access fitting port 12d into water storage tank access port 34, for filling the water storage tank 30 when necessary. Conduit 46 is used to disperse the dry mist and ozone mixture sanitizer in blending chamber 30e to create an ozone-saturated dry mist from water storage tank 30 through water storage tank access fitting 30b and exterior housing access fitting 12b connected to dispersing hose 52. (FIG. 1A). A nozzle 50 (FIG. 1A) may be attached on the end of the hose 52. The hose 52 and nozzle 50 direct the ozone-saturated dry mist on hard and soft surfaces and airborne particles to be sanitized and/or disinfected. Therefore, the system 10 is operated by providing power to air blower 42, ozone generator 44, and the ultrasonic oscillator 40, which has discs suspended underwater, approximately half an inch, that are vibrated to create the dry mist in the blending chamber 30e in the upper portion of water storage tank to create an ozone-saturated dry mist 30. Also shown in FIG. 4 is a drain valve 34 which is connected outside of the exterior housing for emptying the water storage tank 30 of water.

The dispensing and sanitizing method and system can create a sanitizing and/or disinfecting, ozone-saturated dry mist cloud to sanitize and/or disinfect surfaces and airborne particles by creating a dispersed sanitizing evaporating dry mist vapor, super-saturated with ozone. The sanitizing, ozone-saturated dry mist is created using a pressure differential dispersant method. The ozone-saturated dry mist cloud will sanitize and or disinfect hard surfaces and airborne particles and penetrate porous surfaces and ambient air to sanitize and/or disinfect. It kills bacteria and viruses, and other pathogens on hard and soft surfaces and in ambient air.

In some embodiments, a sanitizing apparatus utilizing the components described in FIG. 1B-FIG. 3 can be mounted on a mobile platform, that includes wheels and a frame network with a handle, for manually moving and positioning an entire sanitizing apparatus in various locations, which allows the delivery system to be positioned manually, thereby enabling a technician to thoroughly sanitize and/or disinfect an area with a mobile platform and the ability to manually position the output of the delivery system for sanitizing various surfaces.

There are numerous examples of devices and articles that can be sanitized and disinfected of bacteria and viruses and other biological pathogens using the invention described herein, such as: modes of transportation, delivery vehicles, automobiles, trucks, ambulances, complete airplane including ventilation, trains, buses, spacecraft, subways, cruise ships, and boats; also buildings, including nursing homes, hotels, restaurants, motels, hospitals, office spaces, grocery stores, grocery carts; food processing, buffets and salad bars, kitchens and food preparation service areas; air conditioning ventilation systems, and indoor and outdoor furniture.

The foregoing is considered as illustrative only of the principles of the invention. Numerous changes and modifications will readily occur to those skilled in the art, as it is not desired to limit the invention to the exact construction and operation shown and described, and accordingly, all such suitable changes or modifications in structure and operation which may be resorted to are intended to fall within the scope of the claimed invention.

1. A system for sanitizing and/or disinfecting surfaces and ambient air containing bacteria and/or viruses comprising:
an exterior housing;
an enclosed water storage tank mounted inside said exterior housing and a water supply for storing the water supply for creating ozone-saturated dry mist inside the water storage tank, above a stored water surface level;
an ultrasonic oscillator, mounted on a water float, to position the ultrasonic oscillator below the surface of the water stored in said storage tank to generate dry mist;
an air blower for generating air under low pressure, said air blower having an outlet connected into said water storage tank, above the highest water level permitted in said water storage tank;
an oxygen tank having an outlet;
an ozone generator connected to the outlet of the oxygen tank;
regulator and flow meter connected to said outlet of said oxygen tank; and having an inlet conduit to said ozone generator for regulating the flow of oxygen into the ozone generator;
said water storage tank upper volume above the stored water level providing a blending chamber for said dry mist ultrasonic oscillator output mixture, said air blower air, and said ozone generator output, providing a mixture of ozone-saturated dry mist of a predetermined saturation ratio of dry mist vapor and ozone in parts per million of ozone; and
an ozone-saturated dry mist dispensing outlet and hose for dispensing said ozone-saturated dry mist and ozone super-saturated mixture provided in said water storage tank blending chamber; for sanitizing and/or disinfecting surfaces with ozone.

2. A system as in claim 1, including:
a mobile platform attached to said exterior housing for sanitizing and/or disinfecting surfaces and airborne particles containing bacteria and/or viruses that can be manually moved and positioned around hard and soft surfaces for sanitizing and/or disinfection from the dispensing outlet and hose.

3. A system as in claim 1, including:
said air blower outlet in said water storage tank includes a downwardly positioned airflow diverter for diverting the output airflow downwardly in the direction of the water surface of water contained in the water storage tank and mixture with said dry mist and ozone flow above the surface of the water in the water storage tank creating an ozone-saturated dry mist.

4. A process for sanitizing and/or disinfecting hard and soft surfaces and ambient air containing bacteria and/or viruses comprising steps of:
a) providing a water storage tank that includes a maximum volume of water at a predetermined level in said water storage tank, and a predetermined volume blending chamber above said maximum water level for blending air, ozone, and generating an ultrasonically piezoelectric oscillator dry mist below the water surface and into the blending chamber above said water surface in said water storage tank to create an ozone-saturated dry mist for sanitizing and/or disinfecting soft and hard surfaces and airborne particles;
b) mounting an air blower and its outlet into said water storage tank, the air blower outlet air above the maximum water level permitted in the water storage tank so that in-flowing air from said air blower in said water storage tank is above the water surface level at all times;
c) mounting an ozone generator and its outlet ozone flow generated at a predetermined amount to said water storage tank, below the air blower outlet, above the water surface in said water storage tank, said ozone outlet flow into said water storage tank below the air flow by the air blower into said water storage tank;
d) providing an ultrasonic oscillator below said water surface for generating dry mist and water, positioned in the water stored in said water storage tank, said ultrasonic oscillator mounted and positioned below the water level; and
e) generating dry mist from said ultrasonic oscillator in said water stored in said water tank, into the blending chamber having flowing air from said air blower into said volume above said water stored in said water tank; said incoming airflow directed downwardly toward the surface of said water stored in said water tank, and directing ozone into said water tank above the surface of water stored in the water tank but below the air flowing into said water storage tank, the input of said ultrasonic oscillator generating dry mist, said airflow downwardly into said water storage tank, and said ozone input above said water storage tank water level creating a saturated or supersaturated, ozone dry mist in a predetermined ratio of parts per million of ozone in said ozone-saturated dry mist in the blending chamber above said water level of water in said water storage tank.

5. The process as in claim 4, including the step of:
f) dispensing said ozone-supersaturated dry mist from said blending chamber in said water storage tank onto a hard or soft surfaces and airborne particles outside said water storage tank to remove bacteria and viruses.

6. The process as in claim 5, including the step of:
g) attaching a dispensing hose, exterior to said water storage tank blending chamber, for dispensing the ozone-supersaturated dry mist onto a hard or soft surfaces and airborne particles to remove bacteria and viruses without leaving any water moisture or residue on said hard or soft surface.

7. The process as in claim 4, wherein the dry mist generated in said blending chamber in said water storage tank is 6/10 a liter of water an hour.

8. The process as in claim 4, wherein said ultrasonic oscillator frequency is about 20 thousand Hertz.

9. The process as in claim 4, wherein, said ozone generator produces a precise percentage of 6 grams of ozone per hour for supersaturating said dry mist generated in said blending chamber above the surface of water in the water storage tank to create an ozone saturated or supersaturated dry mist.

10. The process as in claim 4, wherein the ultrasonic oscillator includes a plurality of ceramic discs mounted slightly (one half-inch) below the water level in said water storage tank that generate and produce dry mist particles that are forced into the blending chamber above the surface of the water.

11. The process as in claim 9, where in the air flow into the water storage tank above the surface of the water from the air blower is 12 ft.³ per minute to aid in dispensing the ozone-saturated or supersaturated dry mist from the water storage tank blending chamber.

12. A process for ultrasonically generating an ozone-saturated dry mist, from water, for generating ozone from pure oxygen, for saturating the dry mist with ozone that is dispensed on hard and soft surfaces and airborne particles for sanitizing and/or disinfecting the hard and soft surfaces and airborne pathogens containing bacteria and/or viruses comprising the steps of:
a) generating ultrasonically a dry mist in an enclosed water storage tank partially filled with water, with an ultrasonic oscillator positioned approximately ½ an inch below the surface of the water;
b) providing pure oxygen tank near said enclosed water storage tank;
c) providing an ozone generator having an oxygen input connected to said pure oxygen water tank and an ozone output of the ozone created by the ozone generator connected into the water storage tank above the water level in a predetermined fixed amount of flow rate of ozone O₃;
d) providing a low-pressure supply of airflow from an air blower into said enclosed water storage tank above the level of said water in said water storage tank;
e) saturating with ozone the dry mist generated in said enclosed water storage tank blending chamber above said water with a predetermined amount of ozone in parts per million and
dispensing the dry mist and ozone mixture from said enclosed water storage tank blending chamber to create an ozone-saturated dry mist by a dispensing hose for sanitizing hard and soft surfaces and airborne particles.

13. A process as in claim 12, wherein the saturated ozone in the dry mist constitutes up to 150 parts per million.

14. A process as in claim 12, including the step of:
f) providing an ultrasonic piezoelectric oscillator with a plurality of spaced-apart, ultrasonic-frequency-generating, ceramic discs positioned below the water level, each ceramic disc ultrasonic piezoelectric oscillator having a power source connected in parallel each of the other ceramic discs mounted therein.

15. A process as in claim 12, including the step of:
g) providing said enclosed water storage tank with a flat vertical wall that has the air blower outlet and the ozone generator outlet both mounted to the same wall, said ozone-generator outlet mounted to said water storage tank vertical wall, below said air blower outlet mounted to said water storage tank vertical wall.

16. A process as in claim 12, wherein:
said dry mist developed amounts to 6/10 liters per hour.

17. A process as in claim 12, wherein:
said ozone generated by said ozone generator output is up to six grams per hour.

18. A process as in claim 12, wherein:
said air flow from said air blower output is 12 cubic feet per minute.

19. A system as in claim 1, including:
said ultrasonic oscillator is piezoelectric, and is mounted to said water float, to position the ultrasonic piezoelectric oscillator below the surface of the water stored in said water storage tank; said ultrasonic piezoelectric oscillator including one or more ceramic discs positioned relative to said water float to be submerged below the water storage level at all times for frequency vibration of the water to generate dry mist in said water storage tank.
